(19) United States
(12) Patent Application Publication
(10) Pub. No.: US 2015/0068524 A1
(43) Pub. Date: Mar. 12, 2015

(54) System for a Drug Delivery Device

(71) Applicant: Stefan Mayer, Freiburg im Breisgau (DE)

(72) Inventor: Stefan Mayer, Freiburg im Breisgau (DE)

(73) Assignee: SANOFI SA, Vernier (CH)

(21) Appl. No.: 14/367,508

(22) PCT Filed: Dec. 14, 2012
(86) PCT No.: PCT/EP2012/075625

§ 371 Date: Jun. 20, 2014

(30) Foreign Application Priority Data
Dec. 22, 2011 (EP) 11195313.9

(51) Int. Cl. A61M 15/00

(52) U.S. Cl. CPC A61M 15/0001; A61M 15/0065; A61M 2202/064; A61M 2205/6081; A61M 2205/586; A61M 2209/06
USPC 128/203.15

(57) ABSTRACT

A system comprising an opening aid for opening an inhaler and an inhaler, the opening aid comprising at least one opening member, wherein the inhaler comprises a first and a second exterior part which are connected to one another, wherein the exterior parts are separable by a movement including a relative rotational movement to prepare the inhaler for a dispensing operation, wherein the opening member is configured to be connected to one of the exterior parts in a torque-proof fashion, and wherein the first exterior part is a body comprising a dispensing opening through which a dose of drug can be dispensed from the body and the second exterior part is a cap which is releasably connectable to the body and, when connected to the body, covers the dispensing opening.

### CROSS REFERENCE TO RELATED APPLICATIONS

The present application is a U.S. National Phase Application pursuant to 35 U.S.C. §371 of International Application No. PCT/EP2012/075625 filed Dec. 14, 2012, which claims priority to European Patent Application No. 11195313.9 filed Dec. 22, 2011. The entire disclosure contents of these applications are herewith incorporated by reference into the present application.

### FIELD OF INVENTION

The present disclosure relates to an opening aid for a drug delivery device, e.g. an inhaler, such as a dry powder inhaler.

An inhaler is known from WO 2009/065708 A2, for example.

### BACKGROUND

It is an object of the present disclosure to provide one or more components which facilitate opening of a drug delivery device.

### SUMMARY

This object is achieved by the subject matter of the independent claims. Advantageous embodiments and refinements are subject matter of the dependent claims.

One aspect of the present disclosure relates to an opening aid for a drug delivery devices such as an inhaler. The opening aid comprises at least one opening member which is configured to be connected to an exterior part of the drug delivery device, particularly in a torque-proof fashion.

One aspect of the present disclosure relates to the use of the opening aid for opening an inhaler, the opening aid comprising at least one opening member configured to be connected to an exterior part of the inhaler in a torque-proof fashion.

It is an advantage of the opening aid to aid opening of such drug delivery devices by the user, who may be a private person without clinical background. Providing a separate opening member in addition to a fully functionable drug deliver device may facilitate opening of the device. Opening of the device may be required to make the device ready for operation. A separate opening member is especially suitable for visually and/or physically impaired users of the drug delivery device, i.e. persons who may have difficulties in opening the drug delivery device without the opening member. For example, people having an impaired vision may have difficulties in judging which of a plurality of exterior parts of the device has to be moved with respect to another one of those parts to open the device. Further, people of limited physical strength, like elderly people or children, may be too weak to apply the force which is necessary to open the device. Provision of the separate opening member may hint to the user which portion he has to grip for opening the device, for example due to a marking or a different identification feature highlighting the opening member or due to its mere presence. Further, the opening member may, due to its torque-proof connectability to the exterior part of the drug delivery device assist the user in reliably transferring the force from the user to the exterior part, for example due to grip features provided on the opening member, and, consequently, facilitate opening of the device.

According to the present disclosure a system comprises the opening aid and the drug delivery device. The drug delivery device comprises a first and a second exterior part, wherein these parts are connected to one another, particularly if the device is in a storage state and not operated.

An exterior part may be a part of the drug delivery device which is located at the outside of the device without being covered by additional parts, particularly in the storage state. The opening member is expediently configured to be connected to one of the exterior parts.

In an embodiment, the first exterior part is a body comprising a dispensing opening through which a dose of drug can be dispensed. The body may comprise multiple parts which are, particularly movably, connected to each other. Drug may be provided in the body, e.g. within a powder reservoir retained in the body. The second exterior part may be a cap which is releasably connectable to the body. When the cap is connected to the body, it covers the dispensing opening. In this situation, the device may be in the storage state. When the drug delivery device is opened, the dispensing opening is exposed. Exposing the dispensing opening may be required to prepare the device for a dispensing operation. After the dispensing opening was uncovered, the device may be in the ready-to-use state. Accordingly, in the ready-to-use state, the device is no longer in the storage state. For an inhaler, the ready-to-use state may be a state of the device where the user only has to generate a suction air flow through the dispensing opening in order to inhale a dose of drug. Accordingly, during the transition from the storage state to the ready-to-use state, a dose of drug may be retrieved from the reservoir and be brought into a delivery position, e.g. an inhalation position, by a metering mechanism. The metering mechanism may be operated by the movement of the cap which is required for disconnecting cap and body.

In an embodiment, when the two exterior parts are connected to one another, the drug delivery device may be completely assembled. That is to say, no additional parts are necessary for a proper operation or performance of the drug delivery device in the storage state as well as in the ready-to-use state. The exterior parts of the device may be separable by a movement including a relative rotational movement to prepare the drug delivery device for a dispensing operation. In an advantageous embodiment, the exterior parts are releasably connected by a threaded connection.

In an embodiment, the device comprises a sealing which may be provided between the first and second exterior part, e.g. body and cap. The sealing may be provided by a seal, such as an o-ring, arranged between the two exterior parts. In this way, the interior of the device may be protected against harmful external influence. The sealing may be a moisture tight sealing. When the first exterior part and the second exterior part are disconnected, the sealing may increase the opening force required for opening the device as a sealing usually requires mechanical contact of the involved parts. Accordingly, an initial stiction between the parts may have to be overcome to loosen the sealing engagement. Thus, loosening the sealing requires an initial force that has to be exerted by the user, when the two exterior parts are being disconnected. After the sealing has been loosened during the disconnection of the exterior parts, there is still a force to be exerted, e.g. to operate the metering mechanism. However, this force may be smaller than the initial force. In this regard, a special advantage of the opening member is provided in conjunction with the drug delivery device.

According to the present disclosure a device assembly comprises the system mentioned above. In the device assembly, the opening member may be firmly attached to one of the exterior parts of the drug delivery device, expediently in a torque-proof fashion. The exterior part to which the opening member is attached may be the first or second exterior part. Expediently the opening member is attached to the cap of the device, as the cap is usually the element which is intuitively gripped by a user for opening the device. However, the opening member may nevertheless be connected to the body. An advantage of the torque-proof connection of the opening member and the exterior part of the drug delivery device is that a relative torque applied by the user to the exterior parts via the opening member can be reliably created between the exterior parts, which torque may be necessary to create a rotation which may be required to open the device.

In an embodiment, the torque-proof connection of the opening member facilitates the opening of the drug delivery device which may require a separation of the exterior part from the remainder of the drug delivery device.

In an embodiment, an opening force is the force which has to be applied by the user for opening of the drug delivery device. This force may be applied to the exterior part tangentially with respect to the other exterior part of the drug delivery device.

In an embodiment, the opening member is configured to provide a mechanical transmission ratio such that the opening force is reduced by the opening member. That is to say, the opening force which has to be applied for opening the device without using the opening member is greater than the force which has to be applied when using the opening member. In other words, the application of the opening member may cause the torque exerted onto the exterior part with respect to the other exterior part of the drug delivery device to be increased. As a consequence, elderly users or children are enabled to operate the drug delivery device more easily.

In an embodiment, the opening member comprises a receiving part which is configured to receive the exterior part of the inhaler. Thereby, advantageously a torque-proof connection between the opening member and the exterior part of the drug delivery device may be established. The receiving part may be adapted to cooperate with the exterior part to establish a positive-fit connection or a friction-fit connection between opening member and exterior part. These connections may be torque-proof. The connection between the exterior part and the opening member is expediently configured such that the connection is stable at least up to a force acting on the connection which is equal to the opening force which is required to open the device without using the opening aid.

Referals to the exterior part herein above and below may relate to the first or second exterior part of the device described above.

In an embodiment, the opening member comprises a gripping part which is configured such that the opening force can be applied thereto by the user in order to open the drug delivery device.

In an embodiment, the opening member comprises an outer surface with a coefficient of static friction that is greater than that of the exterior part, in particular when the coefficient of static friction is referred to the same reference material acting on the outer surface, such as the skin of a human hand. An advantage of such an outer surface is that an opening of the drug delivery device by the user is facilitated as the static friction of the hand, when cooperating with the opening member, is enhanced. In this way, the transfer of the opening force from the user to the exterior part may be improved.

In an embodiment, the opening aid or the opening member thereof is configured to be firmly and/or permanently connected to the exterior part of the drug delivery device. Expediently, the opening member is attached to the device. The opening member may be permanently attached to the exterior part during the lifetime of the drug delivery device. The firm attachment may be performed by putting the opening member over one of the exterior parts. To this end the opening member may be structured at the inside of the receiving part as to facilitate the firm attachment. Alternatively the opening member may be attached to the exterior part by heat shrinking or any other suitable techniques.

In an embodiment, the opening member is detached from the drug delivery device after the drug is dispensed. In this case, the opening member may be reused. In another embodiment, the opening member is permanently, particularly, irreleasably attached to one of the exterior parts. When the opening member is firmly connected to the exterior part, the receiving part of the opening member expediently coincides or partly coincides with the gripping part. Particularly, the receiving part and the gripping part may be arranged within the same longitudinal section of the opening member.

In an embodiment, the receiving part comprises a receiver with a wall thickness that determines the transmission ratio. The transmission ratio may be determined by the radial distance of the gripping part from a central longitudinal axis of the drug delivery device. This radial distance may be given by the sum of a radial distance of an outer surface of the exterior part of the drug delivery device from the axis and the wall thickness. As an advantage of this geometry, the reduction of the opening force can be tailored by the wall thickness of the receiver. The transmission ratio and, if applicable, the coefficient of static friction thereby allow the user to open the drug delivery device with less effort as compared to a situation where no opening aid is present. Within this embodiment, the wall thickness may be in the range of few millimeters, e.g. 3 mm or more, or 4 mm or more, but is not restricted to the given values. The opening member may further be configured such that the opening force is reduced by 20% or less, such as 15% or less, and, preferably, by 10% or more.

In an embodiment, the opening member comprises a cover member, in particular a sleeve. The cover member may be configured to be firmly attached to or is firmly attached to the exterior part. The cover member is adapted to be connected to at least partly cover the exterior part of the drug delivery device. The cover member may cover 50% or more of the outer surface of the exterior part. The cover member may be used to protect and/or to individualize the device, in addition to its function as an opening member.

In an embodiment, the opening aid comprises two opening members, e.g. a first cover member and a second cover member. The respective cover member may be a sleeve. The first cover member is configured to be firmly attached to or is firmly attached to the first exterior part and the second cover member is configured to be firmly attached to or it is firmly attached to the second exterior part. The cover members are adapted to be connected to and to at least partly cover two different exterior parts of the drug delivery device. The cover members may be arranged and configured to cooperate with different hands of the user for opening the device, i.e. one member may cooperate with one hand and the other one with the other hand. The wall thicknesses of the cover members, in particular of their receiving parts, may be equal. The respective cover member may envelop a major fraction of the outer surface area of the respective exterior part to which it is connected (see the value recited further above).

The first and second cover member, in cooperation, may cover 50% or more of the total outer surface area of the device, particularly in the storage state. The cover members may be used to protect and/or to individualize the device, in addition to its function as an opening member.

In an embodiment the opening member comprises one or more, preferably visual and/or tactile, identification features. Such an identification feature may include a colour and/or tactile features, e.g. raised structures or Braille letters. The identification feature(s) may indicate the type of drug contained in the drug delivery device and/or a part which the user has to grip in order to open the device. Furthermore, the identification may serve for a personalization of the drug delivery device. The identification feature may further provide a safety aspect, since the likelihood of a confusion of drug delivery devices is reduced which may, e.g., occur in a family with more than one user.

In an embodiment, only one of the cover members comprises an identification feature.

In an embodiment, an identification feature, preferably including a colour and/or one or more tactile features, like e.g. raised structures or Braille letters, may be comprised by both cover members. The cover members can expediently be distinguished by the user, preferably on account of the identification features. The cover members are preferably provided with different identification features, e.g. different colours and/or tactile features. Different identification features indicate to the user, which portions he has to grip to open the device. Especially for users with an impaired vision, such an identification presents a considerable improvement.

One cover member may comprise an identification feature of one type, e.g. visual or tactile, and the other member may comprise an identification feature of the other type, e.g. tactile or visual. The two cover members may comprise identification features of the same type but preferably, the two cover members comprise identification features of different types (e.g. visual and tactile features). In this way, the type of drug contained in the drug delivery device may be indicated by the identification features (e.g. colour) of the first cover member and, e.g. a personalization, may be provided by the identification features (e.g. tactile features) of the second cover member.

In an embodiment, the cover members comprise different wall thicknesses, so as to provide a tactile hint to the user where a joint between the exterior parts is located and, thus, where the opening force has to be applied in order to open the drug delivery device.

In an embodiment, the opening member is a tool which is releasably connectable to one of the exterior parts for opening of the drug delivery device. This embodiment may be alternative to the one where the opening member is permanently connected to the exterior part of the drug delivery device. Such a tool may be used temporarily always before a dose of drug is administered by the user. After the opening of the drug delivery device has been performed, the connection may be released again. In this embodiment, the receiving part is arranged next to the gripping part. In particular the receiving part and the gripping part may be arranged adjacent to each other. The receiving part further comprises a receiver which is configured to establish a positive-fit or a friction-fit to one of the exterior parts in order to open the drug delivery device. As an advantage, such a tool can be easily connected to the drug delivery device in the case of use. The torque-proof connection is thereby guaranteed by the positive-fit or the friction-fit. The receiver may be configured to mate with structures, e.g. raised structures or protrusions which may be comprised by the exterior part. The tool may be adapted to cooperate with the body of the device. The body may be provided with structures to establish a positive-fit or form-fit connection with the receiver. The receiver may be embodied like a socket wrench.

Moreover, the opening force which is applied by the user for opening of the drug delivery device may further be reduced compared to the previously described embodiment, as the tool is not permanently connected to the device and, therefore, does not contribute to the size of the device assembly. Accordingly, size restrictions do apply to the opening aid only to a limited extent.

In an embodiment, the opening member may be configured such that the transmission ratio of the opening force for opening of the drug delivery device is determined by a radial dimension of the gripping part. The radial dimension may be determined by the radial distance between the gripping part and the longitudinal axis of the drug delivery device, when the opening member is connected to the exterior part. To this end the radial dimension may be configured to reduce the opening force for the opening of the drug delivery device by 30% or more, preferably 40% or more, more preferably 50% or more, particularly if the opening member is a tool.

Features which are described herein above and below in conjunction with different aspects or embodiments may also apply for other aspects and embodiments. Particularly, features described with respect to the opening aid may apply for the system and the device assembly and vice versa.

The opening aid also assists closing of the device.

### BRIEF DESCRIPTION OF THE DRAWINGS

Further features and advantages of the subject matter of this disclosure will become apparent from the following description of the exemplary embodiment in conjunction with the figures, in which:

FIG. 1 shows a schematic longitudinal sectional view of a first embodiment of a device assembly according to the present disclosure.

FIG. 2 shows a schematic longitudinal sectional view of a second embodiment of the device assembly according to the present disclosure.

FIG. 3 shows a schematic perspective view of a third embodiment of the device assembly according to the present disclosure.

FIG. 4 shows perspective view of an exemplary embodiment of a system according to the present disclosure.

FIG. 5 shows an exemplary embodiment of the drug delivery device based on a schematic longitudinal sectional view.

### DETAILED DESCRIPTION

Like elements, elements of the same kind and identically acting elements may be provided with the same reference numerals in the figures. Additionally, the figures may be not true to scale. Rather, certain features may be depicted in an exaggerated fashion for better illustration of important principles.

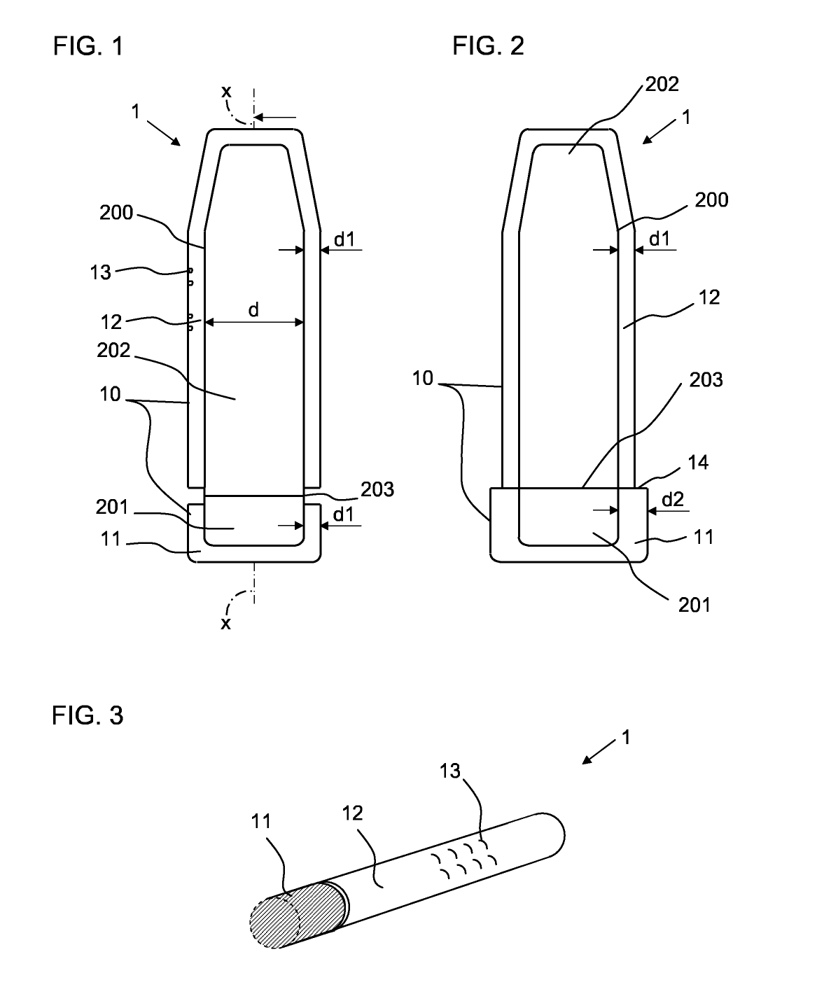

FIG. 1 shows a device assembly 1 according to a first embodiment of the present disclosure comprising a drug delivery device 200, e.g. an inhaler, such as a dry powder inhaler, and an opening aid 10. The opening aid 10 comprises a first cover member 11 and a second cover member 12. The drug delivery device 200 has a cylinder-like shape with an indicated diameter d. The longitudinal axis of the device 200 is indicated with x. The drug delivery device 200 comprises a first exterior part 201 and a second exterior part 202, wherein the said parts 201 and 202 are releasably connected to one another, e.g. by a threaded connection, thereby forming a joint 203. A sealing, e.g. a sealing established by an o-ring, may be provided between the exterior parts. The sealing expediently provides a moisture tight seal.

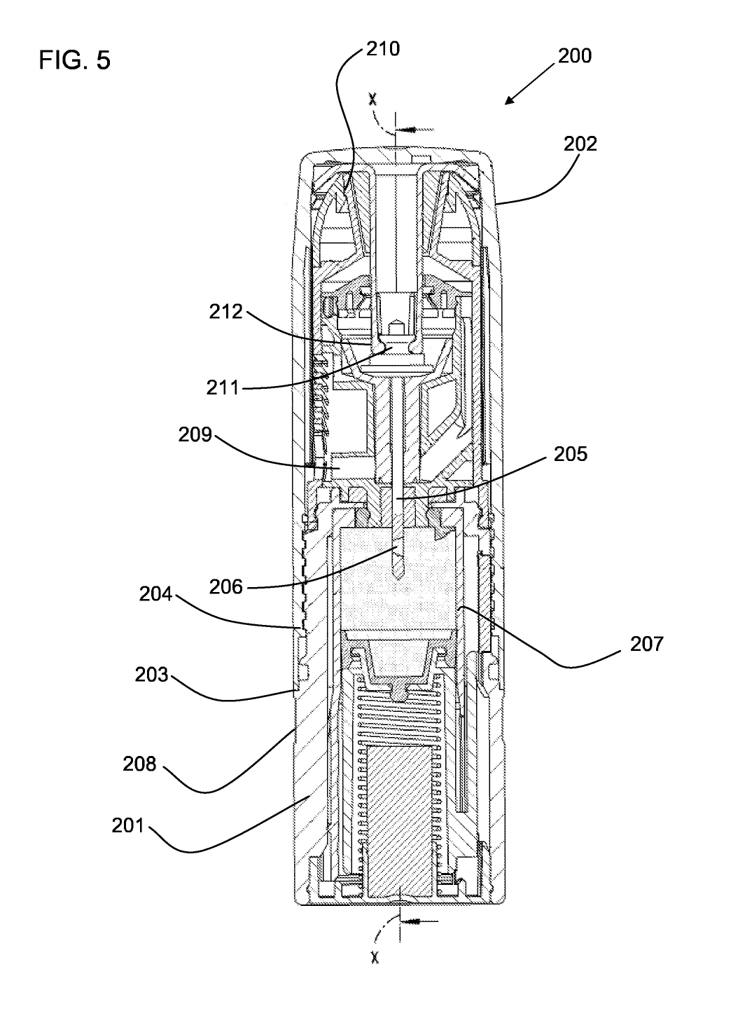

The cover members 11 and 12 are connected in a torque-proof fashion to the exterior parts of the drug delivery device 200, thereby enveloping the drug delivery device 200 such that only a small fraction around the joint 203 is left uncovered, preferably less than 10% of the total outer surface area of the device. The first cover member 11 envelopes the first exterior part 201 and the second cover member envelopes the second exterior part 202. The exterior parts 11 and 12 may be formed by a body and a cap of the drug delivery device 200, respectively. The drug delivery device is described in more detail in conjunction with FIG. 5. The cover members 11 and 12 both have equal wall thicknesses d1 as indicated in FIG. 1.

The respective cover member may be a plastic sleeve pulled over the respective exterior part and, preferably, shrunken onto that part by the application of heat. The cover member and the respective part may be connected by a friction-fit connection in a torque-proof fashion.

The first cover member 11 comprises tactile identification features 13 for an easy identification of the drug delivery device 200 by the user.

The cover members 11 and 12 do assist a user in opening the device, which requires detaching the cap from the body, for example. The assistance may, for example, be provided by a mechanical transmission ratio for lowering the opening force which the user has to exert to open the device. The mechanical transmission ratio for the reduction of the opening force is provided by the wall thickness d1, by which the radial dimension of the device assembly 1 is increased compared to the drug delivery device 200.

The initial threshold torque required for opening of the drug delivery device 200 may amount to e.g. 0.6 Nm. “Initial” relates to the fact that for the said drug delivery devices, there may be an increased initial resistivity against an opening action due to the sealing of the exterior parts 201 and 202.

When assuming a radius of the device of 15 mm, a wall thickness of 3 mm would, for example reduce the opening force by about 15%. In principle, the cover members are suitable to reduce the opening force by 20% or less with a still tolerable wall thickness which does not increase the thickness of the device assembly 1 too much.

To open the device, the user grips the cover members 11 and 12 with different hands and rotates the cap relative to the body, thereby unscrewing the cap from the body. Thus, in this embodiment, a gripping part where the user grips the cover member and a receiving part wherein the part of the device is received, overlap as seen in the radial direction towards the axis or even coincide as seen in the radial direction.

A second embodiment of the present disclosure is depicted in FIG. 2. This embodiment largely corresponds to the one of FIG. 1. In contrast to the embodiment of FIG. 1, the cover members 11 and 12 abut at the longitudinal position of the joint 203.

Additionally or alternatively, the cover members 11 and 12 possess different wall thicknesses d1 and d2. At the longitudinal position of the joint 203 in FIG. 1, the cover members 11 forms a projection 14. The projection 14 results from the different wall thicknesses d1 and d2. This projection provides a tactile identification feature which aids the user in distinguishing the cover members. As a consequence of this tactile information, the user may be guided to grip different cover members with different hands in order to open the device. Alternatively or additionally, different colours may be provided for this purpose.

FIG. 3 shows a third embodiment of the device assembly 1, wherein different colours are used for the cover members 11 and 12. This allows for a personalization and identification of the drug delivery device 200, e.g. of the drug type contained in the device. An additional identification may be provided by tactile identification features 13, which may be implemented by raised structures.

Of course, features from the first to third embodiment may also be combined with one another.

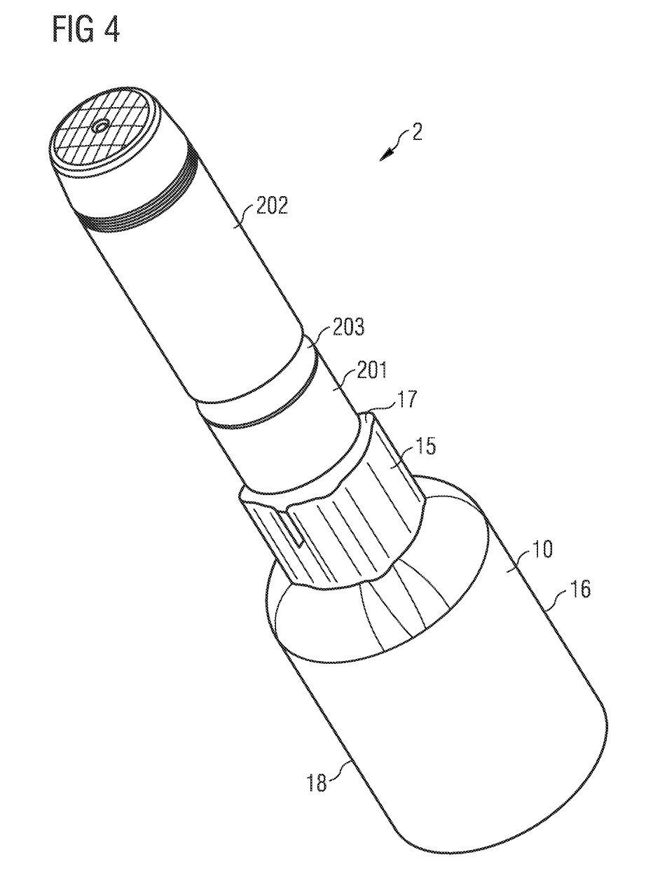

In FIG. 4, a system 2 according to an alternative embodiment of the present disclosure is shown. The system 2 comprises the drug delivery device 200 (see the description above and below) and the opening aid 10. The opening aid 10 comprises a tool 18 which comprises a receiving part 15 and a gripping part 16. The receiving part 15 further comprises a receiver 17 which is configured to form a releasable positive-fit connection with the exterior part 201, e.g. the body. The receiver may be formed like a socket wrench with a structure complementary to a structure on the outer surface of the body to facilitate a positive-fit connection with the body.

The radial dimension of the gripping part 16 may be considerably greater than the one of the exterior part 201 to considerably lower the opening force, e.g. by 30% or more.

FIG. 5 shows the drug delivery device 200, e.g., an inhaler comprising—amongst other parts—a first and a second exterior part 201 and 202 connected by a thread 204 serving for the releasable connection of the exterior parts (201 and 202). The inhaler may have a cylinder-like shape. This device could be used for all of the embodiments described above. The first exterior part 201 is a body and the second exterior part is a cap. The first exterior part 201 includes a dosing pin 205, a dose 206 arranged within a dosing hole of the dosing pin, a drug reservoir 207, a recess 208, a flow channel 209, a dispensing opening 210 and/or a snap receiver 211. The filled drug reservoir 207 may retain a plurality of doses 206 of drug within the body. The drug may comprise a powder. The second exterior part 202 comprises a snap member 212 which is configured to mate with the snap receiver 211. The drug delivery device 200 and its function is similar to the one described in WO 2009/065708 A2, the disclosure content is hereby incorporated by reference into the present description, in particular as far as the operation of the device is concerned.

In order to prepare the drug delivery device 200 for a dispensing operation, the second exterior part 202 is firstly unscrewed from the first exterior part 201 against an initial resistance. The unscrewing further effects the extraction of a dose 206 of drug from the filled drug reservoir 207 via the dosing pin 205, i.e. disconnecting the exterior parts 201 and 202 operates the metering mechanism. This mechanism moves the dose 206 into the region of the flow channel 209 such that it may be extracted from the device by the user when the user generates a suction airstream through the dispensing opening. The force necessary for operating the metering mechanism is expected to be smaller than the one for generating the initial threshold torque. However, this force still has to be exerted by the user once the initial threshold torque has been overcome and can therefore be reduced by the opening aid 10. After the unscrewing of the second exterior part 202, it has to be separated from the first exterior part 201 by disengaging the snap member 212 from the snap receiver 211. As a result the dispensing opening 210 is exposed and the suction air flow generated by the user can retrieve the dose 206 from the dosing pin and the dose may reach the lung of the user. The cover member 11 and 12 (not explicitly shown in FIG. 5) may be connected to the exterior parts 201 and 202 such that a recess 208 of the drug delivery device 200 remains uncovered by the cover members 11 and 12. Such an embodiment would be in accordance with FIG. 1, for example.

The term “drug” as used herein may mean a pharmaceutical formulation containing at least one pharmaceutically active compound, for example for the treatment of obstructive airway or lung diseases such as asthma or chronic obstructive pulmonary disease (COPD), local respiratory tract oedema, inflammation, viral, bacterial, mycotic or other infection, allergies, diabetes mellitus.

The active pharmaceutical compound is preferably selected from the group consisting of active pharmaceutical compounds suitable for inhalation, preferably antiallergenic, antihistamine, anti-inflammatory, antitussive agents, bronchodilators, anticholinergic drugs, and combinations thereof.

The active pharmaceutical compound may for example be chosen from:

an insulin such as human insulin, e.g. a recombinant human insulin, or a human insulin analogue or derivative, a glucagon-like peptide (GLP-1) or an analogue or derivative thereof, or exendin-3 or exendin-4 or an analogue or derivative of exendin-3 or exendin-4;

an adrenergic agent such as a short acting β2-agonists (e.g. Salbutamol, Albuterol, Levosalbutamol, Fenoterol, Terbutaline, Pirbuterol, Procaterol, Bitolterol, Rimiterol, Carbuterol, Tulobuterol, Reproterol), a long acting β2-agonist (LABA, e.g. Arformoterol, Bambuterol, Clenbuterol, Formoterol, Salmeterol), an ultra LABA (e.g. Indacaterol) or another adrenergic agent (e.g. Epinephrine, Hexoprenaline, Isoprenaline (Isoproterenol), Orciprenaline (Metaproterenol));

a glucocorticoid (e.g. Beclometasone, Budesonide, Ciclesonide, Fluticasone, Mometasone, Flunisolide, Betamethasone, Triamcinolone);

an anticholinergic agent or muscarinic antagonist (e.g. Ipratropium bromide, Oxitropium bromide, Tiotropium bromide);

a mast cell stabilizer (e.g. Cromoglicate, Nedocromil);

a xanthine derivative (e.g. Doxofylline, Enprofylline, Theobromine, Theophylline, Aminophylline, Choline theophyllinate);

an eicosanoid inhibitor, such as a leukotriene antagonist (e.g. Montelukast, Pranlukast, Zafirlukast), a lipoxygenase inhibitor (e.g. Zileuton) or a thromboxane receptor antagonist (e.g. Ramatroban, Seratrodast);

a phosphodiesterase type-4 inhibitor (e.g. Roflumilast);

an antihistamine (e.g. Loratadine, Desloratadine, Cetirizen, Levocetirizine, Fexofenadine);

an allergen immunotherapy (e.g. Omalizumab);

a mucolytic (e.g. Carbocisteine, Erdosteine, Mecysteine);

an antibiotic or antimycotic;

or a combination of any two, three or more of the above-mentioned compound classes or compounds (e.g. Budesonide/Formoterol, Fluticasone/Salmeterol, Ipratropium bromide/Salbutamol, Mometasone/Formoterol);

or a pharmaceutically acceptable salt or solvate or esters of any of the above named compounds.

Pharmaceutically acceptable salts are for example acid addition salts and basic salts. Acid addition salts are e.g. a chloride, bromide, iodide, nitrate, carbonate, sulfate, methylsulfate, phosphate, acetate, benzoate, benzenesulfonate, fumarate, malonate, tartrate, succinate, citrate, lactate, gluconate, glutamate, edetate, mesylate, pamoate, pantothenate or a hydroxy-naphthoate salt. Basic salts are for example salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology. Pharmaceutically acceptable ester may for example be acetates, propionates, phosphates, succinates or etabonates.

Pharmaceutically acceptable solvates are for example hydrates.

The scope of protection of the invention is not limited to the examples given hereinabove. The invention is embodied in each novel characteristic and each combination of characteristics, which particularly includes every combination of any features which are stated in the claims, even if this feature or this combination of features is not explicitly stated in the claims or in the examples.

1-13. (canceled)

14. A system comprising an opening aid for opening an inhaler and an inhaler for dispensing a drug, the opening aid comprising two opening members, i.e. a first cover member and a second cover member, wherein the inhaler comprises a first and a second exterior part which are connected to one another, wherein the exterior parts are separable by a movement including a relative rotational movement to prepare the inhaler for a dispensing operation, wherein
the cover members are configured to be connected to one of the exterior parts in a torque-proof fashion, wherein the cover members are adapted to be connected to and to at least partly cover the two different exterior parts of the inhaler, wherein
the first exterior part is a body comprising a dispensing opening through which a dose of drug can be dispensed from the body and the second exterior part is a cap which is releasably connectable to the body and, when connected to the body, covers the dispensing opening, wherein
the opening members comprise one or more visual and/or tactile identification features, wherein the respective cover member is provided with an identification feature, and wherein the identification features are configured such that the cover members can be distinguished by the user, and wherein the cover members are arranged and configured to cooperate with different hands of the user for opening the inhaler.

15. The system according to claim 14, wherein the opening members comprise a receiving part configured to receive the exterior part of the inhaler and a gripping part configured to be gripped by a user such that an opening force, which has to be applied by a user for opening the inhaler, can be applied to the gripping part by the user.

16. The system according to claim 14, wherein the opening member is configured to provide a mechanical transmission ratio such that the opening force is reduced by the opening aid.

17. The system according to claim 14, wherein the opening members comprise an outer surface having a coefficient of static friction which is greater than the coefficient of static friction of the exterior part.

18. The system according to claim 14, wherein the opening aid, particularly the opening members, is configured to be permanently connected to the exterior parts of the inhaler.

19. The system according to claim 15, wherein the receiving part and the gripping part at least partly coincide and the receiving part comprises a receiver with a wall thickness which determines the transmission ratio.

20. The system according to claim 14, wherein the opening members are configured to reduce the opening force for opening the inhaler by 20% or less.

21. The system according to claim 14, wherein the opening member is a tool which is configured to be releasably connectable to the exterior part for opening the inhaler.

22. The system according to claim 21, wherein the receiving part and the gripping part are arranged next to each other and the receiving part of the opening members comprise a receiver configured to establish a positive-fit with the exterior part for opening the inhaler.

23. The system according to claim 21, wherein the opening members are configured such that the mechanical transmission ratio for opening the inhaler is determined by a radial dimension of the gripping part and wherein the opening force for opening the inhaler is reduced by 30% or more.

24. A device assembly comprising a system according to claim 14, wherein the opening members are firmly attached to one of the exterior parts in a torque-proof fashion.

25. Use of an opening aid for opening an inhaler, the opening aid comprising two opening members, i.e. a first cover member and a second cover member, which are configured to be connected to one of the exterior parts of the inhaler in a torque-proof fashion, wherein the cover members are adapted to be connected to and to at least partly cover two different exterior parts of the inhaler, and wherein the opening members comprise one or more visual and/or tactile identification features, wherein the respective cover member is provided with an identification feature, wherein the identification features are configured such that the cover members can be distinguished by the user, and wherein the cover members are arranged and configured to cooperate with different hands of the user for opening the inhaler.

26. System comprising an opening aid for opening an inhaler and an inhaler for dispensing a drug, the opening aid comprising two opening members, i.e. a first cover member and a second cover member, which are configured to be connected to one of the exterior parts of the inhaler in a torque-proof fashion, wherein the cover members are adapted to be connected to and to at least partly cover two different exterior parts of the inhaler, and wherein the opening members comprise one or more visual and/or tactile identification features, wherein the respective cover member is provided with an identification feature, wherein the identification features are configured such that the cover members can be distinguished by the user, and wherein the cover members are arranged and configured to cooperate with different hands of the user for opening the inhaler.
